(19) United States
(12) Patent Application Publication
(10) Pub. No.: US 2008/0064983 A1
(43) Pub. Date: Mar. 13, 2008

(54) Livestock tissue identification method and device

(75) Inventors: Steven Robert Stromberg, Scandia, MN (US); Daniel Ray Hanson, New Richmond, WI (US); Jeffrey Ostberg, Deerwood, MN (US)

Correspondence Address: Mark A. Litman & Associates, P.A.;Suite 205, York Business Center, 3209 West 76th St., Edina, MN, 55435, US

(73) Assignee: Jenrik Ag, LLC

(21) Appl. No.: 11/823,484

(22) Filed: Jun. 27, 2007

Related U.S. Application Data
(60) Provisional application No. 60/843,115, filed on Sep. 8, 2006.

(51) Int. Cl. A61B 10/02; G09F 3/00

(52) U.S. Cl. 600/567; 119/858; 40/300

(57) ABSTRACT

A method and device take samples of tissue from a tissue sample using a tissue punch device. The tissue punch tool has a cutter punch with a cutting end and a force receiving end. A mechanical drive mechanism advances the cutter punch. The cutting end of the punch cutter is positioned into an initial position that is above a tissue from a specifically identified mammal or animal to be sampled. The tissue to be sampled is punched with the tissue punch tool to support a punched tissue sample temporarily within the tool. The supported punched tissue sample is deposited into a single capsule within a clip of multiple capsules that has capsule identification information on the capsules and the clip. The capsule is closed to retain the tissue therein. The capsule identification information and identification information from the specifically identified mammal are combined into a single file and retaining that file in a database.

### BACKGROUND OF THE INVENTION

1. Field of the Invention

The invention relates to, tissue sampling, tissue sample differentiation and marking for testing, and for systems, methods and apparatus using a container for receiving a sample and a means for obtaining said sample. This system may be used with any system for the collection of tissue for sampling and analysis, including spinal samples (for mad cow disease) and ear tag marking systems. The present invention further relates to systems for testing and retaining information on individual livestock and herds of livestock that are applicable for large scale operation enabling tracing of tissue samples back to original livestock sources. The invention enables the use of ear tags for marking animals by withdrawing a biological sample, and then either recording an established animal ear tag identification presented with the animal at the time of sampling or to synchronizing installation of a new ear tag identification number assignment to the sampled animal that corresponds to the tissue capsule receptacle identification number as well as to a method for marking animals with said ear tags.

2. Background of the Art

Ear tags (or ear marks) have been used for a long time for marking and identifying animals, whereby flexible ear tags/ear marks made of plastics, mostly polyurethane or polyethylene, have generally been adopted in the last years. All of said ear tags comprise a mandrel-type plate, also called male part of male flag, and a female part, the counter-plate or female flag. The mandrel-type plate normally comprises a mandrel carrying a tip made of metal or a hard plastics material. The counter-plate comprises an opening, through which the mandrel is pressed when the ear tag is closed after the ear has been penetrated, so that said two ear tag parts are connected with each other. When inserting conventional ear tags, the ear is punctured with the sharp tip of the mandrel, i.e., the tip penetrates through the skin, the cartilage and the skin on the opposite side of the ear.

The tip of the mandrel of the ear tag is normally pushed through the skin and the cartilage, and the skin and the cartilage are ripped up in the form of a longitudinal tear to an extent sufficient to allow the entire head of the mandrel to slip there through. The result is that the mandrel of the inserted ear tag is more or less firmly surrounded by the spread ear tissue which closes elastically.

Ear tags have lately also been used to simultaneously obtain a tissue sample for a later DNA analysis when the ear tag is inserted. A method for withdrawing a tissue sample into numbered sample capsules is, for example, described in DE 197 40 429 A1. According to this method the numbers of the used ear tags and the numbers of the sample container are linked to each other electronically.

An ear tag is additionally described in PCT/EP98/03075, according to which the ear tag and the receiving container are provided with the same coding and the same animal identification number by means of laser or other technologies prior to the issue to the users. This ensures an identity linking of ear tag and tissue sample receiving container without errors and confusions. The hollow tip of the mandrel, which is connected with the mandrel via a predetermined breaking point, takes a sample in the ear, transports the same automatically into the receiving container, separates from the mandrel and hermetically seals the sample in the receiving container.

The container for receiving the sample is normally withdrawn and collected after the ear tag has been inserted. In the European Union it is stipulated by legal regulation that all born calves have to be provided with two ear tags within the first week after their birth. If the ear tags described in PCT/EP98/03075 are now used for said marking, the receiving container of the one ear tag could, for instance, be withdrawn immediately after the ear tag was inserted, whereas the receiving container of the second ear tag remains—connected with the female flag—on the animal. Later, e.g., when the animal is exported, slaughtered or cut up, the second container for receiving the sample still provided on the animal, which contains a sample since the ear tag was inserted, is withdrawn and delivered to the laboratory for the analysis of the same. This procedure saves, in view of the test sample, the recovery and the marking of a new sample and enables the obtainment of an unmistakable second sample, as the same was marked with the ear tag simultaneously, which had been withdrawn already at the beginning of the animal's life, but was preserved in the container 1 for receiving the sample and remained on the animal and is used, for example, only at the end of the animal's life.

U.S. Pat. No. 6,659,338 (Dittmann) describes a method and device for withdrawing biological samples. The device has a receptacle which can receive one or several covers for sample containers, another receptacle which can receive one or several sample containers, and a mechanism. Said mechanism joins the covers and containers together during a working cycle in which the biological sample is withdrawn either through the cover or the sample container to a test capsule.

U.S. Pat. No. 7,198,629 (Brem) describes an ear tag comprising; a mandrel plate (10) having a mandrel (8), a counter-plate (11) having a planar surface for receiving the mandrel (8) wherein the planar surface lacks an opening for the mandrel (8) such that the mandrel (8) penetrates the planar surface during use, a container (1) for receiving a sample, and a hollow tip (4) detachably engaged with said mandrel (8) and having a cut edge for obtaining the sample and sealing the container (1) when the detachable hollow tip (4) is inserted into the container (1) after the sample is obtained, wherein the detachable hollow tip (4) has a cutting edge with at least one undulation.

U.S. Pat. No. 6,509,187 (Brem) describes a device and a procedure for the collection and initial preparation of tissue/blood or other sample of nucleated or DNA-containing cells or cell components for molecular genetic investigation. The invented device for the collection and initial preparation of samples of DNA-containing cells includes a sample receiving container and means for the collection of the sample, which is introduced into the sample receiving container after collection of the sample and seals this tightly. The sample receiving container has a base and side walls, is closed with a easily penetrable lid and has—in an area of the side walls of the container removed from the base—means to secure the introduced sample collection tool; in the container are substances to protect from DNA-degrading enzymes. The tool for the collection of the sample is so formed that on introduction into the sample receiving container it is secured in place by the means in the sample receiving container for securing, and divides the sample receiving container into at least one sample space, which is limited by the base and the side walls of the sample receiving container and the front end of the sample collection tool.

U.S. Pat. No. 6,968,639 (Destoumieux) describes an ear tag for marking animals comprising a female portion including a female head optionally borne by or formed in a first panel, a male portion including a male punch optionally borne by or formed in a second panel, identifying means being borne by at least one of the male or female portions, wherein the male punch is formed by first and second separable punch elements, the first punch element has an end, which in use, becomes housed in the female head to fasten the ear tag onto the ear of an animal and the second punch element forms an organic matter sampling device during the passage of the male punch through the ear of an animal, the second punch element is moveable in an axial channel in the first punch element and is withdrawn from said axial channel through the second panel of the male portion after fastening the tag onto the animal's ear.

U.S. Pat. No. 6,947,866 (Staab) describes an apparatus, and corresponding method, for taking a sample. The apparatus is built around a frame designed to be held in at least one hand. A sample media is used to secure the sample. A sample media adapter for securing the sample media is operated by a trigger mechanism connectively attached within the frame to the sample media adapter. The capture device comprises (a) an apparatus frame sized to be held in at least one hand, (b) a sample media adapter that mechanically holds said sample media, (c) a trigger mechanism connected to said media adapter and adjacent said frame to operate said sample media adapter, (d) means for identifying spatial coordinates of said sample, and (e) means for electronically capturing, processing, and integrating sampling-related data with said spatial coordinates.

Other various tagging systems are shown in U.S. Pat. No. 6,095,915 (Geissler); U.S. Pat. No. 5,741,177 (Roberts); U.S. Pat. No. 5,461,805 (Johnson); U.S. Pat. No. 5,396,898 (Bittmann); and U.S. Pat. No. 5,777,303 (Berney), each of which is incorporated herein by reference for their technical instructions, materials, processes and apparatus.

In spite of the technical variety of ear-tagging systems disclosed and commercially available, there is substantial room for improvement of the devices and the systems.

### SUMMARY OF THE INVENTION

Individual components and systems are described in which a punching device is used to section tissues from livestock (preferably taking ear samples), injecting tissue samples into individual capsules or capsules that may be part of a multi-channeled capsule system, labeling of samples and livestock samples to enable tracing of samples to sources, and multi-channel capsule analysis of encapsulated samples in a matrix analytical system within originally sourced multi-channel capsule collection systems. At least one of, and preferably both of, alphanumeric labeling systems and bar code systems may be used to identify samples and sources. Samples are originally taken with a manual or pneumatically operated tissue punching tool of novel design.

### BRIEF DESCRIPTION OF THE FIGURES

FIG. 1A shows a side view of a manually operated tissue punch gun or sectioning device according to the present invention.

FIG. 1B shows a side view of the device of FIG. 1A in a hand compressed state of punching tissue

FIG. 2 shows a perspective cutaway view of the manual tissue punch device of FIG. 1A associated with a capsule tissue-receptor and cutaway view of a capsule support.

FIG. 3A shows sequentially numbered capsules having an associated and detached series of detachable, single use cutter punch or punch plugs for capsules.

FIG. 3B shows a perspective view of an automatic sample grid alignment of a series of the numbered capsules of FIG. 3A formatted to fit the standard 96-well diagnostic lab plate.

FIG. 3C shows a side perspective view of a system for multi-channel pipetting using the automatic sample grid alignment of FIG. 3B

FIG. 3D shows a section view of the capsules and cutter punches of FIG. 3A, wherein a single cutter punch has been detached and a sample of tissue injected into a first capsule.

FIG. 3E shows an exploded view of a standard 96-well diagnostic lab plate, an 8-capsule array, an 8-cap array, 8 single use punch dies and 8 punch plugs.

FIG. 4 shows a cutaway and exploded view of a capsule top, cap, punch die and punch plug and a cutaway of a tissue-filled capsule with the punch die and punch plug closing an opening on the capsule.

FIG. 4A shows a side view of a tissue-punching system with tissue positioned within the system before punching and above the tissue-receiving capsule array.

FIG. 4B shows a side view of a tissue-punching system with punched tissue positioned within the system after punching and above the tissue-receiving capsule, with tissue deposited into the capsule.

FIG. 4C shows a side view of a tissue-punching system using an array of capsules, and a clip of punch dies and punch plugs.

FIG. 5 shows a tissue punch system with a stabilizing tray for a capsule array, with a sliding cover over the capsule array to allow access to capsules one-at-a-time.

### DETAILED DESCRIPTION OF THE INVENTION

The present descriptions relate to an entire system of tissue sampling technology and should be considered in its most generic sense when reviewing this description. Although elements of the technology for the devices, apparatus, systems and methods may be separately novel, the different elements may also interact within a unified invention. The use of descriptions of specific sizes, materials, process steps and tools should not be read as limiting the scope of the invention, but rather as providing support for the generic concepts described and claimed herein.

Using a multiplicity of attached linear or circular (or arcuate) structure (capsule array) of aligned capsules for receiving the punched tissue samples. The capsules have removable tops that can be replaced after insertion of the tissue. The individual capsules are coded to identify specific sources of tissue and the coding identifiers are stored in a separate database.

The capsules are supported in a support system (e.g., in a fixed, non-moving support of capsule, or in a glide element where the capsules are moved into position to receive the tissue samples. The capsule array may be moved through a punch or pneumatic drive element to keep the array in alignment, or a punch tool may collect tissue and transport the tissue to the capsule array, and the secondary punch or clearing rod may be used to push the tissue from the tool into individual capsules.

Each capsule in the capsule arrays have individual coded identifiers, which may be any unique alphanumeric descriptions. It is preferred that the identifiers are in an easily readable and understandable and logic system, even if bar codes can be scanned and entered without human interpretation. The desired for logical alphanumerics simplifies understanding and retrieval and use by human operators. For example, each boxed set of arrays may have a first identifying code, e.g., Serial Nos. 117,560-117,656 for a set of 12 arrays with 8 capsules in each array. Each array may have a separate identifier, such as 1000-1012 for the twelve arrays, and each capsule in the arrays should have separate unique identifiers, e.g., corresponding to the serial numbers on the box, such as 117,560. Thus, in this formatting, the individual array may have a number of 1002 (or, A1002, or B1002, or μ1002 so that the individual arrays are identified, and then the individual capsule, such as 117,594 is identified. This information can be provided on printed forms provided with the capsule arrays, on software (downloadable on-line or provided with the purchase of a punch and array system), and bar code reading equipment that can download the scanned bar code information from the box containing the array, the individual arrays and/or the individual capsules and enter that information into a database storage system. One bar code scan can enter information on either the individual capsule, a single array of capsules (e.g., 1-20 capsules on a single support), or the entire box of capsules. The alphanumerics on the boxes, arrays and capsules are preferably in a logical notation, such as relatively or precisely sequential. For example, the individual capsules may be numbered 117,560, 117,561, 117,562 . . . etc., and the arrays may be numbers A1000, A1001, A1002, or A1000, B1000, C1000, etc.

The spreadsheet data entry system, in physical format (e.g., typed or hand written entries) or electronic entry, should have the individual livestock identification information added to complete the system. It is desirable that even if hand entered information is initially used, that the complete livestock identification information can be downloaded for ease of access, especially by health officials and regulatory agencies in different counties, states or countries. A standard Excel® spreadsheet can be easily modified to store this data.

The assigned eartag numbers (livestock identifiers) can be pre-entered into the database for the sequential capsule identifiers to assure coordination of data input. The scanner used for the reading of the capsule data (box, array and/or individual capsules) may also be used to read the eartag or other livestock identification information to individually associate the livestock identification information with the capsule information. The livestock information and capsule information may be manually entered through the device (as a primary or backup function), scanned from bar codes, or scanned for RFID or any other machine readable storage (e.g., even smart chips, optical recordings, and the like).

If an array of tissue punches (alternatively referred to herein as punch plugs) are used, the individual tissue punches may have individual plungers attached to the support for the array. A primary drive plunger or outer driver will press the attached plunger at about the same time that the primary driver forces the tissue punch through the tissue. The secondary driver (alternatively referred to herein as the clearance rod) can then push the attached (now separated) plunger to press tissue into the capsule, without the clearance rod contact the tissue directly.

Most of the elements of the supports and plungers and tissue punches that are reused should be periodically cleaned (e.g., washed and/or disinfected) to remove intersample contamination or buildup that might promote bacterial growth.

After the tissue is delivered to the individual capsules, the caps of the capsules should be inserted and secured (e.g., snapped or otherwise affixed) to the capsule top. The caps should be removable, preferably in a reusable way so that multiple samples or diagnostics can be taken from a single tissue sample, yet the original remaining sample stored).

Capsules are preferably sterile before tissue insertion, although this is not important for DNA analysis, as long as the capsule is free of DNA containing material.

The identification system for use with the present technology can be used in a variety of manners in conjunction with the hardware described in the practice of the present invention. One mode of operation is to provide boxes of arrays of capsules, each box separately identified, each array separately identified and each capsule separately and distinctly identified. The box may be scanned in, and this information will identify each of the arrays and each of the capsules. Alternatively, each array may be scanned in, and this will identify each of the capsules provided in that array. To securely provide this function, it will be desirable to have technology that assures that arrays and capsules will be used in an appropriate (e.g., sequential or otherwise corresponding to the order in which individual capsules are filled with portions of the tissue samples) manner. For example, when the box is opened, arrays should be removable only in a specific order, as by having a single opening in a case through which only a single array at a time can be removed, so that only a first array (the first array in the scanned information) can be used. Additionally, it is desirable that each array of capsules can be inserted into a support device or accessed by the user or readily identified by the user as being in a particular order, without having to necessarily read all of the information on each individual capsule. This can be exemplified by the perspective image of a tissue sampling system and capsule array 118 support system 196 in FIG. 5.

In FIG. 5, the array 118 of individual capsules 120 is supported on an array support or accessing device 196. The device is shown with a number of desirable features. A sliding cover 198 moving along directional line 194 is provided so that only a single capsule at a time can be accessed through port 220 with a top opening 224 that is slid into position over any individual capsule that is to be used for delivery of a punched portion of a tissue sample. An array 118 engaging or directional input control system is shown comprising a tooth 190 and an opening 192. There is no tooth on a far side of the array 118, so a user will always know that there is a single elative position for each array to be inserted into the support 196. Additionally or alternatively a series of registration lines 194a (or registration colors or numbers) may be provided on both the support 196 and on the array 118 so that a user can immediately see if the array 118 has been inserted into the support 196 in a proper orientation and sequence of capsules. In this manner, accurate positioning and therefore accurate reading of arrays can be provided.

The scanner used to read the capsule and animal identification code and information may be multifunctional, being able to read bar codes, RFID, provide wired or wireless communication to a data source, be able to record scanned data and download that data through a linkage (e.g., pin linkage, USB port, cable, or other memory link-up) to a database. The database will then correlate scanned animal identification (which may be scanned in advance or after each individual tissue sample is deposited into a capsule) with each capsule identification. Additional identification may be included with the animal and capsule identification, such as present location of the scanning (e.g., specific farm or slaughterhouse), date, personnel taking the sampler and the like. Because each type of information may be provided by a different information source, the multifunctionality is desirable. The scanner may require authorization to be used, as by RFID recognition of authorized personnel or at least proper identification of personnel so that a higher level of confidence can be provided in the samples.

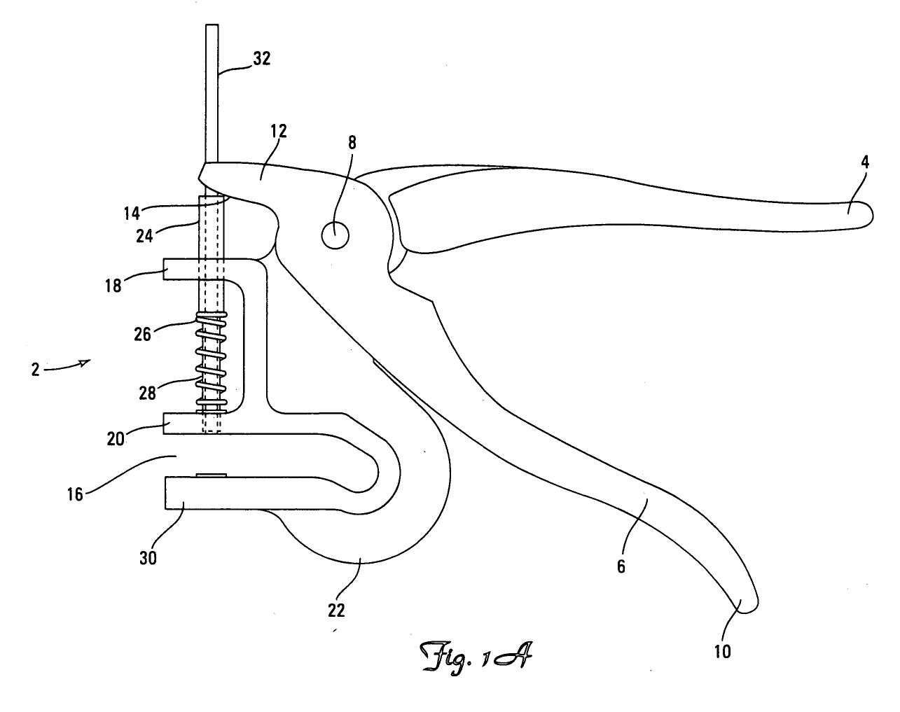

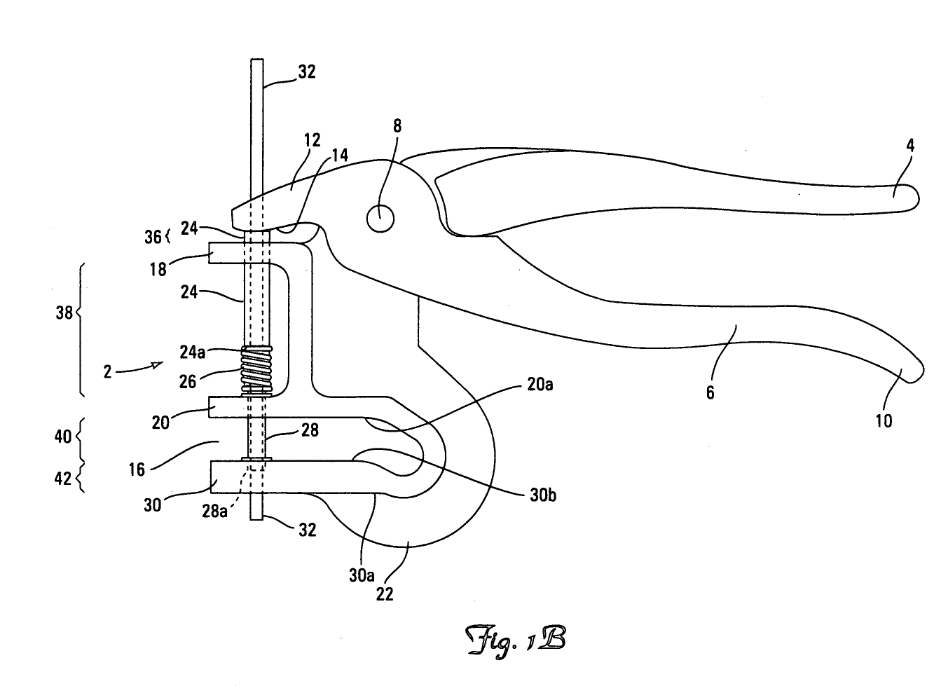

A punch and tagging system may be used according to teachings of the prior art, in conjunction with inventive systems and processes of the present invention, or an improved system according to the present technology may be used. An improved tissue punch device 2 and system according to the present technology is shown in FIG. 1 to which reference will now be made.

The punch device 2 of FIG. 1A comprises a rigid palm grip extended arm 4, a pivoting or relatively moveable finger grip arm 6 shown joined to the rigid palm grip 4 by a pivot rod 8. The finger grip arm 6 has a gripping end 10 and a levered end 12 with a levered action surface 14. Fixed to the palm grip 4 is a capsule supporting structural element or frame 22 which provides an open area 16 for insertion of tissue to be punched, such as an ear section (not shown) and to allow a punch end 28 to pass through the tissue sample, carry tissue sample to a capsule (not shown) and allow retraction of the punch stem 24. The upper punch stem 24 and punch end 28 passes through openings or channels in upper guide plate 18 and then downward through lower guide plate 20 during punching of tissue into capsules. The punch stem 24 and clearance stem 32 is provided with a retraction element (here shown as spring 26) to steady the punch stem 24 and punch end 28 and to help retract the punch stem 24 and punch end 28 after tissue has been punched and ejected from below base plate 30 into a capsule (not shown). Slideably engaged with the punch stem 24 is a clearance stem 32 which moves within a channel in the punch stem 24 and clearance stem 32 to assure that maximum amounts of tissue are ejected from the punch device 2 and that replaceable single use cutter punches (not shown) are pressed firmly into a capsule (not shown).

FIG. 1B shows a side view of the device of FIG. 1A in a hand compressed state of punching tissue. Similar parts from FIG. 1A have similar numbers in FIG. 1B. The respective dimensions of some elements and parts can be significant as will be described, but the dimensions and relative size of other elements may be fundamentally insignificant within reasonable limits. A gap 36 is shown between the levered action surface 14 and the top of the upper guide plate 18, for example. There may be no gap, a slight gap or a relatively larger gap, as long as the levered action surface 14 forces the top of the punch stem 24 so that the punch end 28 passes through the base plate 30 at least at the lowest gap spacing 36 to assure punching of tissue between gap 16. However, as apparent as the variation in the spacing within gap 36 may appear, there is significant complexity in the spacing 38 between the upper guide plate 18 and the lower guide plate 20 (which may also be referred to as a plunger strip, which holds the cutter punches and through which the punch stem is guided and progresses), and the length of the punch stem 24 and the punch end 28. The path length of movement of the bottom 24a of the punch stem 24 and the bottom 28a of the punch stem 28 is the same, as they are connected, although the punch end 28 may be relatively moveable with respect to the punch stem 24 in a concentric or telescoping manner. During the entire path of levered movement of the levered action surface 14 of the levered arm 12 of the finger grip arm 6, the bottom end 28a of the punch stem 28 must pass along a path length that extends at least adjacent to or from the bottom surface 20a of the lower guide plate 20 at least well into the base plate 30 so that the end 28 of the punch stem 28 passes into the top surface 30b of the base plate 30 and preferably extends through the bottom surface 30a of the base plate 30. This is required so that the end 28a of the punch stem or plunger 28 does not block the gap 40 where the tissue is to be punched and the punch stem or plunger 28 passes down carrying a tissue sample to be ejected from the base plate 30. At the same time, the movement of the punch stem 24 is restricted by a lower limit of the bottom 24a of the punch stem 24 within approximately gap 38 between the upper guide plate 18 and the lower guide plate 20, while at he same time allowing compression of the spring 26 within that same gap 38. As the clearance stem 32 slides within the punch stem 24 and punch end 28, that element is not restricted bt the various gaps 36, 38 and 40. The length of movement of the various elements may therefore be described as follows.

Even though the levered arm pivots and moves in an arcuate manner, there is a vertical component (the vertical component is parallel to the axis and length of the cutter punch stem and clearance stem) in the movement of the levered action surface 14. That vertical component path length must be at least as long as the width of the gap 40 between lower guide plate 20 and the base plate 30 and is preferably at least as long as the gap 40 and the thickness 42 of the base plate 40. More preferably, the vertical component path length of the levered action surface 14 is greater than the gap 40 and the thickness 42 of the base plate 40. This length of movement is desired so that total length of movement of the bottom 28a of the punch end 28 is sufficient to pass from a stationary position of the bottom 28a within, above or at the top of the gap 40 to a final extended position at the top surface 30b, within base plate 30 or below the bottom 30a of base plate 30.

Methods and devices useful within the generic scope of the present technology may be described as a method of providing tissue samples from live animals (especially mammals, but also avians and aquatic animals such as fish) for tracing a source of the live tissue back to the live animals or live animal source. The method may comprise:

providing an attached linear or circular array of sets of a) cutter punch or external driver and b) internal clearance stem (e.g., an elongate stem, or secondary driver that pushes tissue lodged within the cutter punch interior) and which are moveable with respect to the punch plugs or disposable cutting dies, the linear or circular array being removeably attached to a support. The linear strips or array or the circular array may be in supports with multiple (e.g., 2, 3, 4, 6, 8, 10 or more) punch plugs carried on a support, especially with a clearance stem acting as a support for the punch plug, the punch plug and/or the clearance plug being severably attached to the support. The attachment may be severed by extension of the punch driver or rod or punch end pressing against or impacting an edge or surface of the punch plug or the clearance plug.

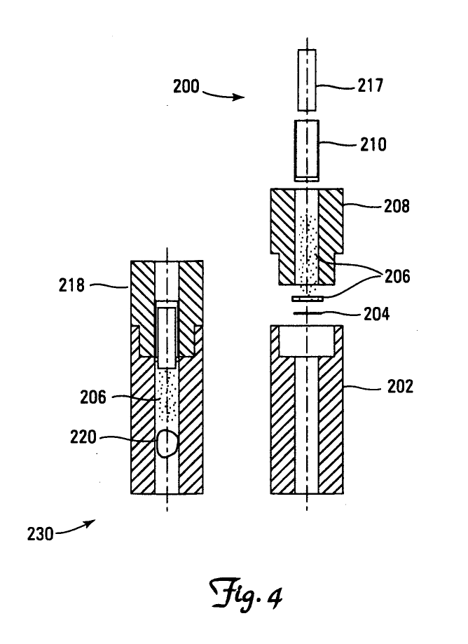

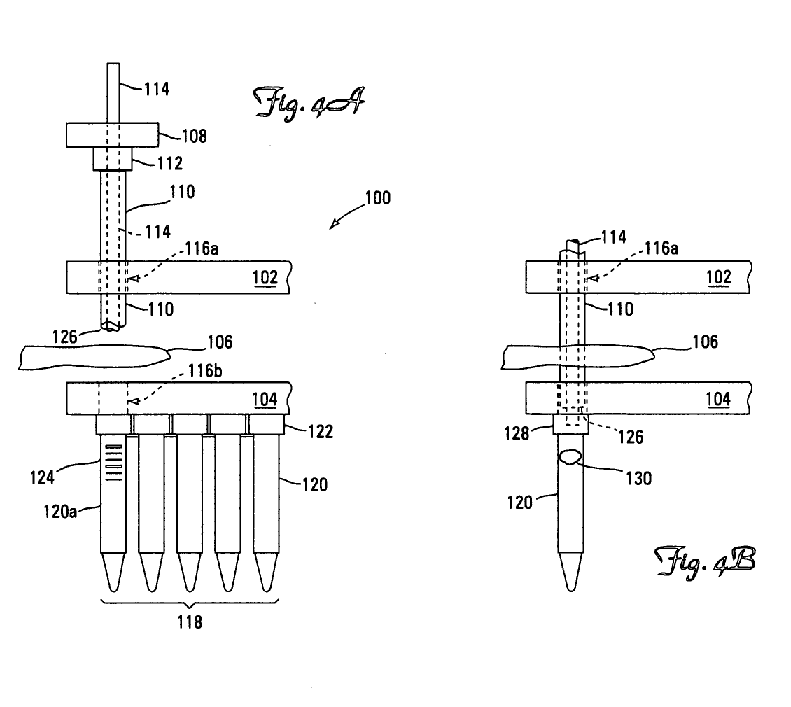

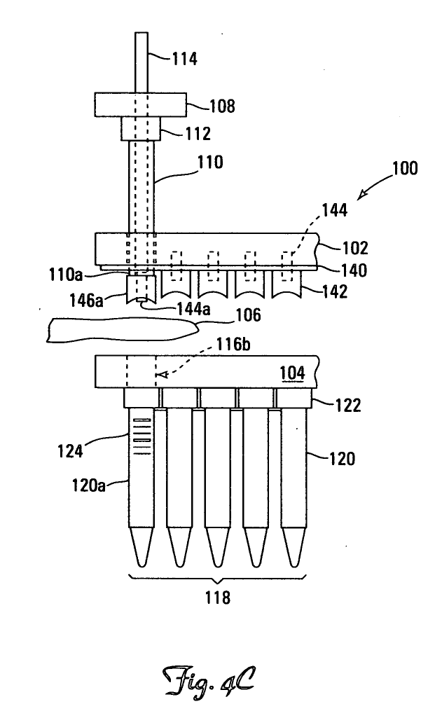

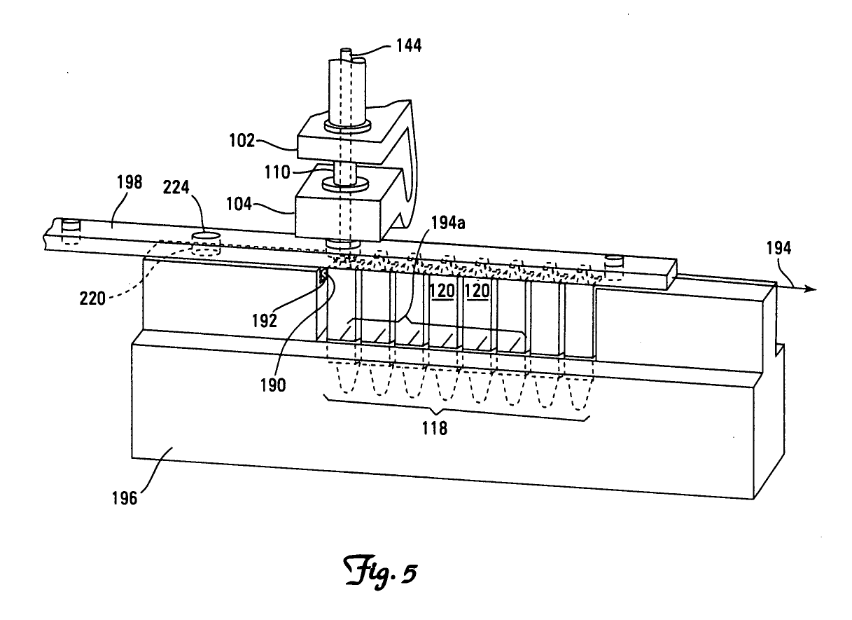

There are alternative methods of providing the tissue samples and placing them into the individual sequences of capsules. Above was described the use of a hand tool in which individual samples were cut (punched) from tissue and carried to the capsules, or arrays of capsules may be fed (as with a liner clip, arcuate clip or circular clip of sequential capsules that may be fed or moved one-at-a-time into position for use with the hand punch. A stationary tissue punch, where animals may be led to the punch, their ears (or other extremities) placed in the punch area, and the samples taken may also be performed. A view of FIGS. 4A and 4B can assist in an appreciation of that format of providing tissues. Again, the use of the identification information and the arrays of capsules with individual capsule identifiers thereon is a preferred embodiment.

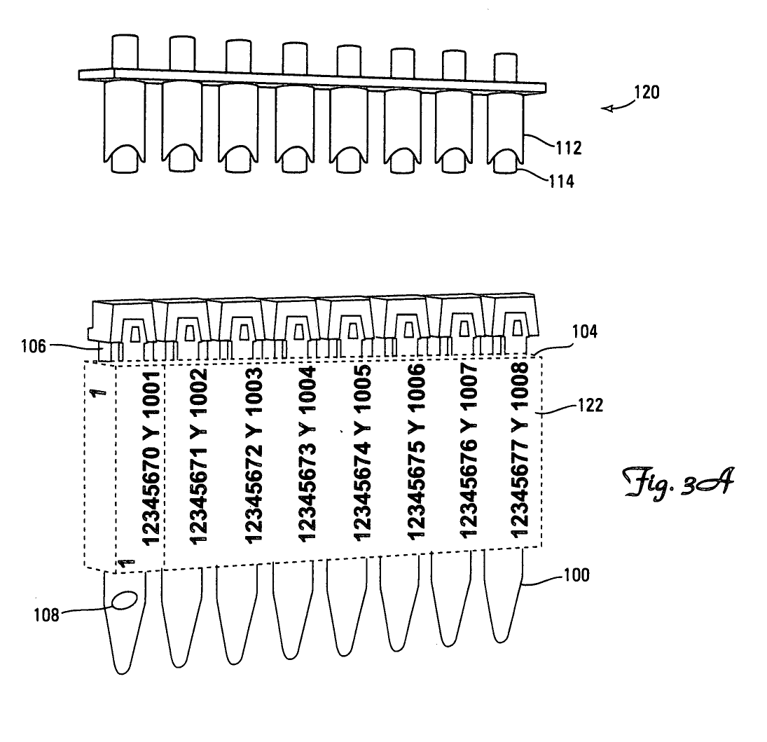

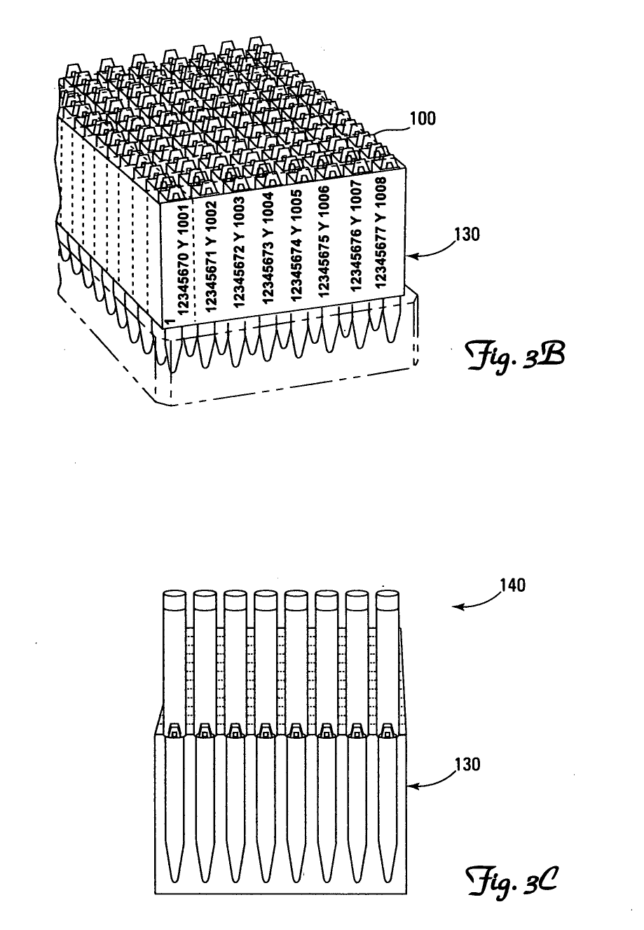

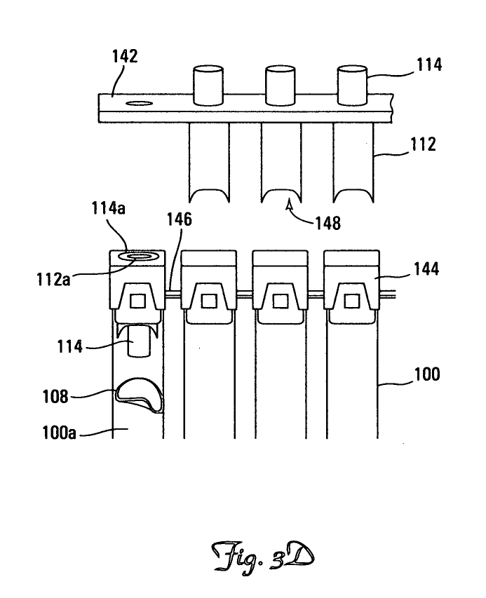

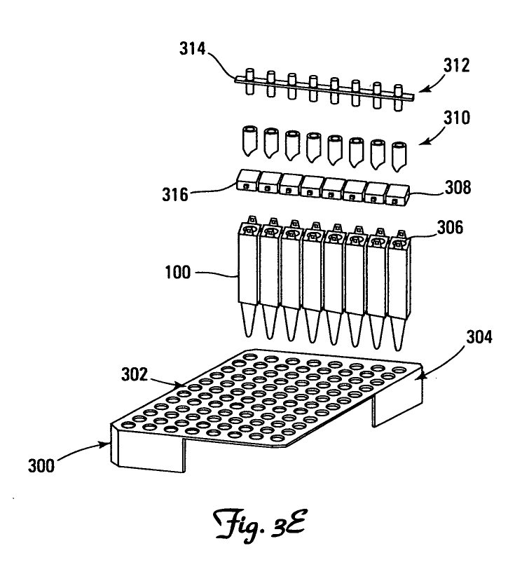

FIG. 3A shows sequentially numbered 122 capsules 100 having an associated and detached series 120 of detachable, single use cutter punch dies 112 or punch plugs 114 for the capsules 100. Snap supports 106 are shown as well as a tissue sample 108 within a capsule 100. A continuous label sheet 104 wraps around at least a portion of the capsules 100.

FIG. 3B shows a perspective view of an automatic sample grid alignment 130 of a series of the numbered capsules 100 of FIG. 3A formatted to fit the standard 96-well diagnostic lab plate.

FIG. 3C shows a side perspective view of a system 140 for multi-channel pipetting using the automatic sample grid alignment 130 of FIG. 3B.

FIG. 3D shows a section view of the capsules 100 and cutter punches (dies) 112 and [punch plugs 114 on a support 142 of FIG. 3A, wherein a single cutter punch die 112a and single punch plug 114a has been detached and a sample of tissue 108 injected into a first capsule 100a. The lead cutting edge 148 of a punch plug 112 is shown as well as a cap 144 fit to the top of the capsule 100. A connecting strip 146 between capsules is shown, which may be permanent or rupturable.

FIG. 3E shows an exploded view of a standard 96-well diagnostic lab plate 300 having receptacle holes 302 for capsules and a stabilizing base 304, with an 8-capsule 100 array 306, an 8-cap 316 array 308, 8 single use punch dies 310 and 8 punch plugs 312 with a support strip 314.

FIG. 4 shows a cutaway and exploded view of a capsule to 202p, cap 208, punch die 210 and punch plug 217 and a cutaway of a tissue (220)-filled capsule 230 with the cap 218 closing an opening 206 on the capsule 230. Tissue stabilizing material 206 supported in the cap 208 by a small cap 204 is also shown as an alternative.

FIG. 4A shows a complete punch system 1000. The punch system 1000 is shown with a top guide plate 102 and a bottom guide plate 104. A tissue sample 106 has been placed between the top guide plate 102 and the bottom guide plate 104. A motor or drive system 108 (e.g., pneumatic motor, step motor, electric motor, etc.) is provided to give force to movement of an exterior or outer driver or cutting die 110. An optional moveable contact element 112 is shown. Within the outer driver 110 is the extendable rod or inner driver 114 that is used to clear tissue from within the outer driver 110. Guide paths or ports 116a and 116b are shown in the top guide plate 102 and the bottom guide plate 104. The outer driver 110 and the inner driver 114 are shown slightly protruding from below the top guide plate 110 for convenience. The outer driver 110 and inner driver 114 need not extend below the top plate 102 but may be in an uppermost retracted position within the upper guide port 116a. It may be positioned slightly above the top plate 102, but this is les preferred as minor misalignment of the drivers 110 and 114 might cause the system 100 to stick or wear more rapidly.

A series or array 118 of sample capsules 120 are shown with a common support 122 below the bottom guide plate 104. A first capsule 120a having bar code identification information 124 (for the individual capsule and/or for the entire series 118 of capsules 120 is positioned directly below the lower guide plate 104 port 116b and in line with the outer driver 110 and the inner driver 114 paths of movement. The motor 108 will drive the outer driver 110 through the tissue sample 106 and carry cut tissue (not shown) through the guide port 116a into the aligned individual capsule 120a. A leading sharp cutting edge or die blade 126 is provided to assist in cutting a smooth and consistent sample from the tissue sample 106. It is not generally necessary to have a replaceable leading edge 126 to prevent contamination, especially with respect to DNA contamination in samples. However, it is an option to provide easily replaceable segments at the cutting edge 126 (e.g., that snap on and snap off) between sampling efforts. Otherwise, simple washing or sterilizing between taking of samples may be desirable for testing for microbial contamination of samples to prevent cross-contamination of samples.

FIG. 4B shows a next sequence in events during a cutting process. Like numbers to those in FIG. 4a indicate like elements. The outer driver 110 has been driven by the motor (not shown) through the tissue sample 106, and into the lower guide port 116b in the bottom guide plate 104. The inner driver 114 has been extended slightly farther than the outer driver 110 to extend below the bottom guide plate 104, into anad through a cap 128 on the individual capsule 120 and drive a cut tissue portion 130 into the individual capsule 120, which capsule 120 is then sealed to retain the cut tissue portion 130 for later analysis if needed.

FIG. 4C shows a further alternative construction that also has a bearing on the use of the hand held punch device of the previous discussion, even though FIG. 4C relates to a stationary tissue sampling system 200. Again, all like numbers of elements refer to like elements in previous figures. In FIG. 4C, a support 140 for a series of single use cutting punches or dies 142 with single use secondary drivers 144 (which also act to plug caps 122) are provided, The outer driver 110 has been slightly advanced to separate a first secondary driver 144a and a first single use cutting punch 146a towards the tissue sample 106. Not shown is that after punching a portion from the tissue sample, and carrying that portion through the cap 122 and depositing the portion into the capsule 120a, it is the separated secondary driver 144a that will press the portion into capsule 120a and the separated single use cutting punch 146a will, in combination with the separated secondary driver 144a penetrate the cap 122 and assist in closing and sealing the cap 122. Additional closure may be desirable, such as a second cap (not shown) snapped onto or other wise secured to the first cap 122.

In performing the method, an individual one of the sets is presented to a punch mechanism (e.g., the punch driver or rod and/or punch end) which severs the association of the individual set in the array, and the most forward (usually lowest) edge of the punch plug slices or punches through the tissue and punches out the tissue sample. The punch mechanism linearly directs a single set of a cutter punch and a internal clearance plug through the live tissue, carrying punched tissue within a forward end of the cutter punch plug. The punch mechanism severs attachment of the single set from the support. The punch mechanism directs the punch plug, clearance plunger or stem and punched tissue into an individual retaining capsule. The user provides information on both a particular animal from which the live tissue was removed and on the capsule that identifies the tissue in the capsule as coming from the particular animal so that a source of the tissue in the container can be traced. The tissue is accurately matched up with alphanumeric or automatically readable (e.g., bar code) identifying information that can be distributed among a tag or tattoo on the animal, recorded data at the livestock facility and on the tissue sample. This type of information is generally referred to in the art as trace-back information, and is used to associate the biological sample in a specific capsule to the originating farm and/or slaughterhouse and to identify a specific animal from which the sample was taken. The use of labels on the arrays of capsules, preferably with pre-assigned traceback information (such as sequential and preferably unique identification information), on a separate sheet that may be stripped from the sheet and applied to the individual capsules, and arrays of capsules. This information may optionally be provided into permanent records at the animal source site or a central clearing bureau is a convenient format for accessing the identification information. It is also possible for the information to be provided as alphanumeric, bar code or other digital format, magnetic stripes, smart chips, RFID transceivers and antennae or any other information providing formats. The method is easily performed where a tag on the particular animal contains data that can be related directly to data on the capsule containing tissue from that particular animal.

The technology may also be described as a system for the reception of tissue samples and identification of sources of tissue samples,. The system may have an array of at least four containers having one open end; a support permanently securing the array of containers together; the open end of each container facing in a same direction; the array containing unique identification information for each of the at least four containers; the unique identification information being machine readable; and at least four open end caps to securely close the open end. The system may have each array with machine readable information that identifies individual arrays. The machine readable information that identifies individual arrays may also includes machine readable information identifying individual containers. The containers are preferably less than 1 cm in diameter and less than 8 cm or less than 7 cm in length. The machine readable information may appear on one side of the array and visually readable information may appears on the same or an opposite side of the array. The system preferably has at least 8 containers are permanently attached in a linear, arcuate or circular array of containers. By “permanently” it is meant that even though individual capsules or containers may be broken off the array, there is sufficient stability in the array under normal handling of the array, such as loading the array into the hand-held device, loading it onto a carrier, affixing a label thereto and the like. Visible readable identification appears on the array to identify the individual containers and a separate recording sheet contains parallel readable identification information for the individual containers. The system may also containing visually readable information concerning identification of a specific livestock. The information concerning identification of the specific livestock may also be present on a tag permanently affixed to a single unique animal, and the separate recording sheet comprises an electronically stored virtual sheet or a physical sheet with printing thereon. The information concerning identification of the specific livestock may also be present on the individual container and that individual container contains an actual sample of livestock tissue therein A box containing multiple systems may comprising at least four arrays and at least one sheet containing the parallel readable identification for each array and may comprise at least four arrays and at least one sheet containing the parallel readable identification for each array. The system may further comprise a processor contains the electronically stored virtual sheet and has a communication link to a central governmental database that stores information regarding livestock tissue samples.

The method is preferably performed where each individual retaining capsule is physically attached within an at least linear array or circular array of individual retaining capsules that are physically attached to at least one adjacent individual retaining capsule and each individual retaining capsule is associated with the data relating to the capsule. After severing attachment of the single set, the clearance plug may be extended to project punched tissue deeper into the individual retaining capsules that contain the tissue samples. By using the cutter punch and clearance stem or plunger in this system, the punch mechanism elements may be prevented from direct contact with tissue and contamination between samples can be avoided.

Another description of a method according to the present technology for taking a sample of tissue from a live tissue sample using a tissue punch tool comprises:

providing a tissue punch tool comprising a cylindrical punch rod or punch driver or drive arm (that presses down on the cutter punch and drives it through the tissue), a mechanical drive mechanism for advancing the cylindrical punch rod, an upper punch rod guide plate having an opening guiding the (for example, cylindrical or other geometric cross-section) cutter punch rod or punch driver through the upper guide plate, a lower punch rod or punch press guide plate having an opening guiding the cylindrical punch driver or rod through the lower guide plate, and a base plate having an opening therein of sufficient size as to allow penetration and passage by the cylindrical punch driver, cutter punch and/or punch rod.

positioning a punch end of the cylindrical punch rod into an initial position that is above, level with or below a surface of the lower punch rod guide plate so that a live tissue on s live mammal can be inserted into a gap below the end of the cutter punch rod and above an upper surface of the base plate;

placing live tissue or other verifiable tissue from a specific animal to be sampled within the gap;

advancing the punch end of the cylindrical punch rod from an initial position to a most extended position through the live or available tissue to punch a sample and carry a punched sample through the base plate opening;

depositing the tissue sample into a capsule and providing an identification of tissue source on the capsule;

wherein the mechanical drive mechanism for advancing the cylindrical punch rod has a range of motion, when in contact with an upper contact point with the cylindrical punch rod, that extends a distance equal to that from the initial position of the punch end of the punch rod to the most extended position of the punch end of the punch rod beneath the base plate. The method may be practiced where advancing the punch end is done against a resilient force such as a compressive element (spring or elastic or foam or rubber) or pneumatic element (e.g., compression valve or chamber) and the like, so that the full extension of the punch movement recovers from stored energy. The method is preferably practiced as described herein with a supported series (linear array or circular array) of disposable or single use cutter punches supported below the punch end of the punch rod and above the live tissue. The arrays may be slid into an active position, supported by structure on the drive mechanism, such as snapped into place, supported on glides that position individual punch cutter punches in line with the punch??? rod and the individual capsules. The method removes a single use or disposable cutter punches by the cylindrical punch rod and the single use punch cutter punch carries tissue into the capsule. The individual capsule is preferably a first of a series of capsules in an attached linear array, and after removal of the single disposable, one-time use cutter punches, the linear array of capsules is reoriented with respect to the base plate so that when the method for taking a sample of tissue from a live tissue sample using a tissue punch tool is repeated, tissue is carried into a second capsule adjacent the first of a series of capsules. After removal of the single-use cutter punches, the supported series of disposable punch plugs are reoriented to position a second removable punch plug along a line of movement for an advancing punch rod. A tag with an animal identification number is attached to an animal from which the live tissue was removed and a second label having information correlating the animal with corresponding tag numbers is associated with the capsule. The attachment of the tag may be contemporaneously with, prior to or subsequent to punching of the tissue sample from the animal. When the field lab report form is added into a data base, such as as national livestock database used by the Food and Drug Administration and the animal is entered into the food chain or shipped to a different livestock facility, the identification data is entered into a semi-permanent system (e.g., storage may be maintained for a fixed number of years until the data is no longer necessary, such as 1, 2, 5, or 10 years or more. The tissue sample can be stored and catalogued (according to identification information and class of livestock) for efficient retrieval at a later date in the event that retrieval will be required for live animal or meat source traceback information to match requested information on a particular animal or herd of animals

The device for punching tissue from a live animal into a capsule has been described in greater detail elsewhere, but may be further described as comprising:

a cutter punch or rod,

a mechanical drive mechanism for advancing the cylindrical punch rod (which may be a plunger or hammer device),

an upper cutter punch guide plate having an opening guiding the cylindrical punch rod through the upper guide plate,

a lower punch rod guide plate having an opening guiding the (preferably cylindrical) cutter punch rod through the lower guide plate, and

a base plate having an opening therein of sufficient size as to allow penetration and passage by the (preferably cylindrical) cutter punch or punch rod;

a punch end of the cutter punch or punch rod is positioned into an initial position that is above, level with or below a surface of the lower punch rod guide plate providing a gap below the end of the cylindrical punch rod and above an upper surface of the base plate;

the punch end of the cylindrical punch rod being advanceable from an initial position to a most extended position through live tissue in the gap to punch a sample and carry a punched sample through the base plate opening; and

an attached linear array of capsules having an alphanumeric, bar code, smart chip or RFID identification of tissue source on the capsule. The mechanical drive mechanism for advancing the cylindrical plunger or driver rod or punch rod has a range of motion, when in contact with an upper contact point with the cylindrical punch rod, that extends a distance equal to that from the initial position of the punch end of the punch rod to the most extended position of the punch end of the punch rod beneath the base plate. It is preferred that there be a linear array of disposable punch cutters on a support provided between the punch end of the punch rod and an opening of the capsule. The mechanical drive is preferably supported on a hand-held device and may be manually actuated and manually powered or manually activated and powered by an electrical or pneumatic power source.

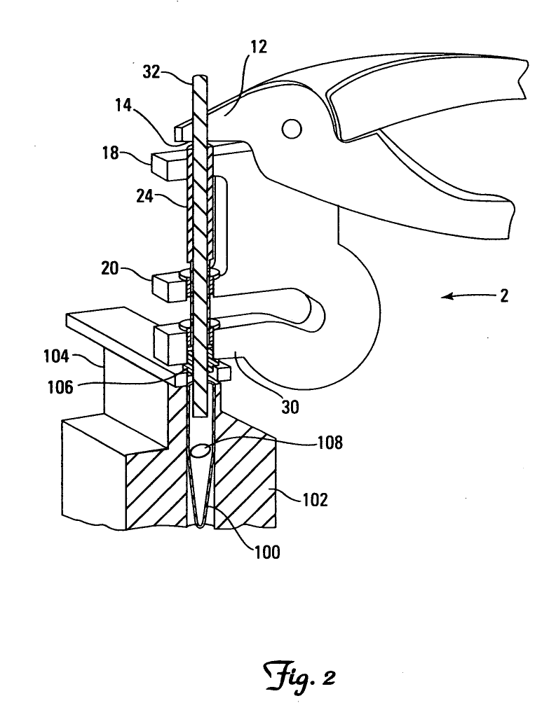

FIG. 2 shows the manual tissue punch device 2 of FIG. 1 associated with a capsule tissue-receptor or capsule 100 and cutaway view of a capsule support 104 holding a capsule or capsule 100 acceptor array. A cap 106 is shown on the individual capsule 100 which has pushed a sample 108 deep into the capsule 100. A preferred embodiment is shown in FIG. 3E where the cap 316 from the cap array 308 is independently or collectively removable from theb array of capsules 100. Rather than puncturing a cap and filling the puncture hole with a single use punch and plug stem, a pre-existing hole 306 in the individual capsules in the array 100 so that a single use cutting punch 310 will cut through tissue (not shown) and carry the tissue into the hole 306 and the punch plug 312 would be pushed by a clearance stem (not shown) deeper into the hole 306 and then an individual cap 316 would be secured to the top of the individual capsules or the collective array to secure the tissue within the individual capsules 100. The stem plug 312 may or may not be used in this embodiment, and the plug may or may not have to remain within the punch die 310. This system offers significant advantages in the reduction of punch forces necessary, without having to punch through a cap (even with a serrated opening), ease of access to tissue within the capsule by simple removal of the cap, and the like. By providing a removable cap, more flexibility in the use of the device is allowed to serve broader markets and livestock management situations. Since the caps are molded together in strips of eight or ten or twelve or more, rapid removal and replacement of 8 or 10 caps at a time is a fast procedure. Lab access to the biological sample or pre-packaged agents are also easier with the simple cap closure design.

The tissue collection capsules may be molded together in groups of eight or more and of a dimension and size that conforms or is compatible with the standard 96-well laboratory plate in common use today for disease and DNA diagnostics. In addition, the conical shape of the tip may conform to the standard lab thermal cycler block that is used in DNA extraction. This provides time-saving convenience in the DNA lab and permits DNA extraction using the ProSampler capsule as the vessel and eliminating the need to remove the biological sample.

By molding the capsules together in groups, one single label can be attached to the strip that will uniquely identify each tissue collection sample capsule. Because of the 8 or more grouping, the unique numbers assigned may also be sequential. This is advantageous because it aids in the organization of samples as they come into the lab and moved into the 96-well lab tray. A custom-designed Excel® file organizes the capsules in the test kit as presented to the field and follows the samples to the lab for easy organizing and maintaining accuracy of individual animal I.D. match-up to the biological sample. Instead of having to enter the I.D. of every individual test tube (either by manual entry or bar code scan), a single capsule I.D. number entry of the first sample will automatically knock in the following sequential numbers into the Excel file. A bar code option that reads the first capsule in each strip will also provide further optional convenience in the lab, and enter the entire series of capsules into the laboratory file.

The Excel® file sets up a 96 sample test kit on a digital file that prints out on an 8½×11 inch page (or size selectable page, such as A4 paper) for a field report or lab report. The report can be packaged with each 96 sample test kit and be pre-printed with the unique, sequential capsule I.D. numbers, group by 8 or 10 or more (or less) row clips of sample collection capsules. In the adjacent field, the individual animal I.D. from which the sample is taken and deposited into the capsule is recorded as a cross-reference in the data base to the pre-assigned capsule number. This assists in maintaining the accuracy of the tissue identification match-up with the animal from which the sample was taken. In addition, the laboratory will use the next adjacent field to assign an identification number of the 96-well plate that will handle and potentially store the biological samples. Every unique sequentially numbered sample collection capsule is also pre-assigned a grid position in the 96-well plate in order to quickly locate the sample on the plate. Optionally, field use can include scanning the radio frequency identification (RFID) of the individual animal directly into the Excel file corresponding to the collection capsule I.D. that is receiving the animal sample. This will allow optimum speed and accuracy in collecting and handling data.

FIG. 3A shows sequentially numbered capsules 100 having an associated and detached series of detachable, single use cutter punches 120 for capsules. A linear array 120 of punch plugs 112 and clearance plugs 114. The clearance plugs 114 are shown extending out of the punch plugs 112, but ordinarily the punch plugs are preferred to extend past the ends of the clearance plugs 114. Labels 122 containing unique capsule identification information (e.g., 12345678 Y 1001, identifying a unique capsule (e.g., 1001) within unique array 12345678) are provided (preferably by automatic assignment and advancement of the array of cutter punches and capsules or otherwise provided on the capsule array. It is also possible to have an automated reading system that reads the label on the capsule, has user input available for entering livestock identification information and records the combined data electronically and can later provide that combined source of information. A scanner can be associated with the system that can be used to read each label and scan the tag on the animal, combine that information into a single file and later transmit that single file and collection of files for the entire array to a memory to store and later access the data. The clearance plugs 114 are shown extending out of the punch plugs 112, but ordinarily the punch plugs are preferred to extend past the ends of the clearance plugs 114. This is an optional structure and the clearance plugs may extend slightly beyond thepunch plugs as the punch forces will cause the initial movement of the clearance plugs remain stationary since they can freely move within the punch plugs. Labels 122 containing unique capsule identification information (e.g., 12345678 Y 1001, identifying a unique capsule (e.g., 1001) within unique array 12345678) are provided on the capsule array.

FIG. 3B shows a perspective view of an automatic sample grid alignment 130 conforming to the standard 96-well diagnostic lab plate allowing multichannel pipetting of liquids in and out of the capsules of a series of the numbered capsules 100 of FIG. 3A.

FIG. 3C shows a perspective view of a system 140 for multi-channel pipetting using the automatic sample grid alignment 130 of FIG. 3C.

FIG. 3D shows a section view of the capsules 100 and punch plugs 112 of FIG. 3A, wherein a single cutter punch 112a has been detached and a sample of tissue 108 injected into a first capsule 100a. The cutter punches 112 are supported by a support 142 which also is shown carrying the clearance plugs 114. A linear array 120 of punch plugs 112 and clearance plugs 114. The ends 148 of the punch plugs 112 are shown with a cutting edge to facilitate puncture of the tissue. Supporting elements 146 are shown securing the linear array 144 of capsules 100. A single cutter punch 112a and clearance plunger 114a are shown closing the opening on the capsule into which they have been inserted and the tissue sample 108 deposited.

FIG. 3E shows a capsule receptor tray 300 conforming to standard 96-well diagnostic lab plate, thus establishing individual grid position assignments to each individual capsule and therefore each individual sampled animal having individual rows of receptacles 302 for individual capsules 100 to be inserted. The individual capsules are shown in a capsule array 304 having individual openings 306 at the top. A fixed (secured) array of caps 308 are shown in an exploded perspective from the capsule array 304 as are a series of punch plugs 310 and an attached clip 314 of clearance plugs 312 which also act to close the top of the capsules 100 in combination with the punch plugs 310.

There are numerous strategic and structural advantages to the system described in the practice of the present technology. Capsule design and volume capacity permits a variety of different formats and recovery of tissue for biological sampling including tissue, blood, milk, and swabs that have been used to collect saliva, blood, semen as well as other bodily fluids for DNA analysis, disease diagnostics or long term storage archiving of biological samples to allow DNA confirmation for traceback capability.

Capsule design and volume capacity permits pre-packaging of a variety of dry or liquid media for the purpose of preserving the biological sample such as with anti-microbial agents that might include a desiccant, salt or iodine mixed with dimethyl sulfoxide (DMSO) or agents that facilitate disease diagnostics such as phosphate buffered saline solution (PBS) for Mad Cow Disease (BVD); Antigen Capture ELISA testing; or a liquid fixative agent such as formalin for immunohistochemistry (IHC). Capsule design and volume capacity allows flexibility for the end user to package their choice of media for preserving or any other such handling requirement of the biological sample.

A capsule design permits a “clip-loaded” group of at least eight, ten, twelve or more tissue collection devices into a clip-fed manual or pneumatic punching tool for convenience and speed in the field. As described above, these arrays or clips may be used individually or fed as a clip into a hand-held device or a stationary punch device.

The structure allows pre-assignment of unique and sequential numbering of individual but attached sample collection capsules. This pre-identification formay facilitates use of a custom-designed Excel® software file (or any other spreadsheet format) to read a single bar code (or single manual entry, or other mechanically readable entry system, as described herein) identifying the first sample collection capsule and thus automatically entering in the following sequential numbers, thus eliminating the labor-intensive requirement of entering or bar code scanning individual sample collections.

Use of a customized Excel® software file automatically assigns plate identification and precise grid position/location on the plate for cataloging, storing and easy retrieval of individual sample(s). The scanning from the data on the box or array or capsule is entered into the spreadsheet, which then allows the tissue identification information to be appropriately entered with, for example, the next open capsule data location.

One desirable format and structure and design for the capsule array conforms to the standard (12×8 row) 96-well lab plate, permitting multi-channel pipetting of fluid in or out of 8-10 capsules at a time as opposed to requiring pipetting of fluids in or out of individual test tubes.

The formatting and structure of the systems of the present technology permits DNA extraction processes with tissue remaining in the ProSampler™ capsule. There is no requirement to remove and handle each tissue sample, for example. Using conical tip ends on the collection capsules to conform to the standard lab thermal cycler block used for the purpose of DNA lab processing facilitates more general use of the capsule structure in a wide variety of technical fields.

In conjunction with use of the customized Excel® spreadsheet file, the system simplifies populating 96-well lab trays with multiple client samples in order to optimize diagnostic testing expenses as a result of unique, sequential numbering of attached sample capsules. The system overall allows improved accuracy of individual animal identification match-up with the biological sample from field collection to lab analysis of high thru put volume biological samples. The system allows a small foot-print for long term tissue storage (up to two million samples in a 10×10 room). The small capsule size reduces storage volumes and allows use of existing storage and staging equipment and can reduce paper files by using the customized spreadsheet systems.

In addition to punching tissue, the capsules may contain any biological sample (e.g., blood, biological swab, saliva, semen, milk, amniotic fluid, serum, and the like) and benefit from the capsule array structure and information system provided in the practice of the present technology.

What is claimed:

1. A method for taking a sample of tissue from a live tissue sample using a tissue punch tool comprising:
providing a tissue punch tool comprising a cutter punch having a cutting end and a force receiving end, a mechanical drive mechanism comprising a plunger for advancing the cutter punch, an upper cutter punch guide plate having an opening guiding the cutter punch through the upper guide plate, a lower cutter punch guide plate having an opening guiding the cutter punch through the lower guide plate, and a base plate having an opening therein of sufficient size as to allow penetration and passage by the cutter punch;
positioning the cutting end of the punch cutter into an initial position that is above, level with or below a surface of the lower cutter punch guide plate so that a live tissue on a live mammal can be inserted into a gap below the cutting end of the cutter punch and above an upper surface of the base plate;
placing live tissue to be sampled within the gap;
the plunger advancing the cutting end of the cutter punch from the initial position to a most extended position through the live tissue to punch a sample and carry a punched sample through the base plate opening;
depositing the tissue sample into a capsule and providing a tissue identification on the capsule;
wherein the mechanical drive mechanism for advancing the plunger has a range of motion, when in contact with an upper contact point with the cylindrical punch rod, that extends a distance equal to that distance from the initial position of the force receiving end of the cutter punch to the most extended position of the force receiving end of the cutter punch when the cutting end of the cutter punch is beneath the base plate.

2. The method of claim 1 where advancing the punch end is done against a resilient force.

3. The method of claim 1 wherein a supported series of disposable punch plugs are supported below the punch end of the punch rod and above the live tissue.

4. The method of claim 3 wherein a single one-time use cutter punch is removed by the cylindrical punch rod or driver rod and carries tissue into the capsule.

5. The method of claim 4 wherein the capsule is a first of a series of capsules in an attached linear array, and after removal of the single use cutter punch disposable punch plug, the linear array of capsules is reoriented with respect to the base plate so that when the method for taking a sample of tissue from a live tissue sample using a tissue punch tool is repeated, tissue is carried into a second capsule adjacent the first of a series of capsules.

6. The method of claim 4 wherein after removal of the single disposable cutter punch punch plug, the supported series of disposable plunger punch plugs are reoriented to position a second removable cutter punch punch plug along a line of movement for an advancing plunger punch rod.

7. The method of claim 3 wherein an ear tag identification tag is independently attached to an animal from which the live tissue was removed and a second label with animal identification information on the tag correlating the animal identified on the tag to the capsule.

8. The method of claim 4 wherein a tag with a matching identification number on a first label information is attached to an animal from which the live tissue was removed and a second label with information correlating the animal identification number to the capsule.

9. The method of claim 5 wherein a tag with a first label information is attached to an animal from which the live tissue was removed and a second label with information correlating the animal to the capsule.

10. The method of claim 6 wherein a tag with a first label information is attached to an animal from which the live tissue was removed and a second label with information correlating the animal to the capsule.

11. A method of providing tissue samples from live animals for tracing a source of the live tissue back to the live animals, the method comprising:
providing an attached linear or circular array of sets of a) cutter punch plugs and b) internal clearance plungers plugs that are moveable with respect to the punch plugs, the linear or circular array being removably attached to a support;
providing individual ones of the sets to a punch mechanism;
the punch mechanism linearly directing a single set of a punch plug and a internal clearance plug through the live tissue, carrying punched tissue within a forward end of the punch plug, and severing attachment of the single set from the support;
the punch mechanism directing the punch plug, clearance plug and punched tissue into an individual retaining capsule; and
providing information on both a particular animal from which the live tissue was removed and on the capsule that identifies the tissue in the capsule as coming from the particular animal so that a source of the tissue in the container can be traced.

12. The method of claim 11 wherein a tag on the particular animal contains data that can be related directly to data on the capsule containing tissue from that particular animal.

13. The method of claim 11 wherein each individual retaining capsule is physical attached within an at least linear array or circular array of individual retaining capsules that are physically attached to at least one adjacent individual retaining capsule and each individual retaining capsule is associated with the data relating to the capsule.

14. The method of claim 13 wherein each capsule and identification information for each capsule is assigned a grid position in the standard 96-well plate for cataloging, storing and retrieving the capsule.

15. The method of claim 11 wherein after severing attachment of the single set, the clearance plug is extended to project punched tissue deeper into the individual retaining capsule.

16. A device for punching tissue from a live animal into a capsule, the device comprising:
a cylindrical punch rod,
a mechanical drive mechanism for advancing the cylindrical punch rod,
an upper punch rod guide plate having an opening guiding the cylindrical punch rod through the upper guide plate,
a lower punch rod guide plate having an opening guiding the cylindrical punch rod through the lower guide plate, and
a base plate having an opening therein of sufficient size as to allow penetration and passage by the cylindrical punch rod;
a punch end of the cylindrical punch rod positioned into an initial position that is above, level with or below a surface of the lower punch rod guide plate providing a gap below the end of the cylindrical punch rod and above an upper surface of the base plate;
the punch end of the cylindrical punch rod being advanceable from an initial position to a most extended position through live tissue in the gap to punch a sample and carry a punched sample through the base plate opening; and
a linear array of attached capsules having an alphanumeric, bar code, smart chip or RFID identification of tissue source on the capsule;
wherein the mechanical drive mechanism for advancing the cylindrical punch rod has a range of motion, when in contact with an upper contact point with the cylindrical punch rod, that extends a distance equal to that from the initial position of the punch end of the punch rod to the most extended position of the punch end of the punch rod beneath the base plate.

17. The device of claim 16 wherein a linear array of one-time use, disposable punch plugs on a support is provided between the punch end of the punch plunger rod and an opening of the capsule.

18. The device of claim 17 wherein the mechanical drive is supported on a hand-held device.

19. The device of claim 18 wherein the mechanical drive is manually actuated and manually powered.

20. A method for taking a sample of tissue from a live tissue sample using a tissue punch comprising:
providing a tissue punch tool comprising a cutter punch having a cutting end and a force receiving end,
a mechanical drive mechanism advances the cutter punch;
positioning the cutting end of the punch cutter into an initial position that is above a tissue from a specifically identified mammal to be sampled;
punching tissue to be sampled with the tissue punch tool to support a punched tissue sample temporarily within the tool;
depositing the supported punched tissue sample into a single capsule within a clip of multiple capsules and providing capsule identification information on the capsule;
closing the capsule to retain the tissue therein; and
combining both the capsule identification information and identification information from the specifically identified mammal into a single file and retaining that file in a database.
